(19) United States
(12) Patent Application Publication
(10) Pub. No.: US 2008/0300529 A1
(43) Pub. Date: Dec. 4, 2008

(54) Skin Care Method and Kit Using Peltier Thermoelectric Device

(75) Inventor: Fred M. Reinstein, Beverly Hills, CA (US)

Correspondence Address: Law Office of John K. Pike, PLLC, 2121 Eisenhower Avenue, Suite 200, Alexandria, VA, 22314, US

(73) Assignee: Farr Laboratories, LLC, Los Angeles, CA (US)

(21) Appl. No.: 12/133,105

(22) Filed: Jun. 4, 2008

Related U.S. Application Data
(60) Provisional application No. 60/941,929, filed on Jun. 4, 2007.

(51) Int. Cl. A61N 1/30

(52) U.S. Cl. 604/20

(57) ABSTRACT

One embodiment provides a method of treating the skin or body part, comprising contacting the skin or body part with a composition; and contacting the composition and heating and/or cooling the skin or body part with a thermoelectric Peltier device. Another embodiment provides a skin care kit, which comprises a composition suitable for application to the skin; and a thermoelectric Peltier device configured to contact the composition and to heat and/or cool the skin. Other embodiments are also disclosed.

### CROSS REFERENCE TO RELATED APPLICATIONS

This application claims priority to U.S. Provisional Application Ser. No. 60/941,929, filed Jun. 4, 2007, the entire contents of which being hereby incorporated by reference.

### BACKGROUND

### Field of the Invention

The present invention relates to skin and/or body care.

### BRIEF DESCRIPTION OF THE DRAWINGS

FIG. 1 presents a schematic illustration of one embodiment of the invention.

FIG. 2 presents an illustration of another embodiment of the invention.

FIG. 3 presents photographs of a female subject before and after treatment with an embodiment of the invention.

### DETAILED DESCRIPTION OF THE SEVERAL EMBODIMENTS

One embodiment provides a method of treating the skin or body part, comprising: contacting the skin or body part with a composition; and contacting the composition and cooling the skin or body part with a thermoelectric Peltier device.

Another embodiment provides a method, comprising: contacting the skin with a composition; and contacting the composition and cooling the skin with a thermoelectric Peltier device.

Another embodiment provides a skin care kit, which comprises: a composition suitable for application to the skin; and a thermoelectric Peltier device configured to contact the composition and to cool the skin.

Another embodiment provides a method of treating the skin or body part, comprising: contacting the skin or body part and cooling the skin or body part with a thermoelectric Peltier device.

Another embodiment provides a skin or body care kit, comprising: a composition suitable for application to the skin or body part; and a thermoelectric Peltier device configured to contact the composition and to cool the skin or body part.

Another embodiment provides a method of treating the skin or body part, which comprises: contacting the skin or body part with a composition; and contacting the composition and heating and/or cooling the skin or body part with a thermoelectric Peltier device.

Another embodiment provides a method of treating the skin or body part, which comprises: contacting the skin or body part and heating and/or cooling the skin or body part with a thermoelectric Peltier device.

Another embodiment provides a skin or body care kit, which comprises: a composition suitable for application to the skin or body part; and a thermoelectric Peltier device configured to contact the composition and to heat and/or cool the skin or body part.

Another embodiment provides a thermoelectric Peltier device, which comprises: at least one surface configured to contact the skin or body part and heat and/or cool the skin or body part.

In one embodiment, the kit and method herein provide a skin care or other benefit including one or more of reducing or eliminating the appearance of fine lines and wrinkles, retarding or reducing the noticeable effects of skin aging, retarding or reducing the appearance of dark circles around the eyes, retarding or reducing the appearance of puffiness around the eyes, treating or reducing the effects of at least one of the following: skin wrinkling, sagging, dryness, irritation, inflammation, swelling, aging, photodamage, bruising, dandruff, acne, keratosis, eczema, flakiness, age spots, hyper-pigmented skin, inflammatory dermatosis, psoriasis, calluses, sunburn, candida, thrush, burn injury, atopic dermatitis, rosacea, fibroblast collapse, low collagen production, skin blemishes, corns, boils, bursas, styes, or a combination thereof.

In another embodiment, the kit and method herein provide a skin care or other benefit including one or more of aiding in improving skin texture, improving skin smoothness, improving skin firmness, improving skin tone, promotion of smooth and supple skin, improving skin elasticity, moisturizing skin, improving the radiance, clarity and general youthful appearance of skin, improving skin color, improving skin hydration, increasing flexibility, firmness, smoothness, suppleness and elasticity of the skin, improving a skin connective tissue, re-stretching collapsed fibroblasts, interfering with collagen breakdown, enhancing collagen production, enhancing elastin production, cooling the skin, cooling elastin and/or collagen, reduce swelling of bursas, exfoliating, detaching and shedding of superficial epithelial cells, improving the thickness of skin, enhancing skin repair and healing, or a combination thereof.

In another embodiment, the kit and method herein provide a benefit not limited to the skin. The kit and method herein provide a therapeutic benefit not limited to the skin.

In another embodiment, the kit and method herein provide a benefit including one or more of treating or reducing pain, discomfort, or irritation in one or more parts of the body, or reducing swelling in one or more parts of the body. In another embodiment, the kit and method herein provide a benefit including one or more of treating or reducing pain, discomfort, or irritation in one or more parts of the body caused by, or reducing swelling in one or more parts of the body caused by any one or more of surgical procedure, bodily injury, bruising, hair waxing or removal, menopause, hot flashes, headache, nausea, laser treatment, microdermabrasion, exfoliation or other skin treatment, carpel tunnel syndrome, temporomandibular joint (TMJ) disorder, sports injury, overuse injury, muscle soreness, and the like, and combinations thereof. In another embodiment, the kit and method herein provide a benefit to both the skin and another part of the body.

In one embodiment, the thermoelectric Peltier device may be suitably used to treat a sty proximal to the eye. In this embodiment, the thermoelectric Peltier device contacts and heats the sty. A moist pad, cover, sock, or sleeve may be used over the skin-contacting surface of the thermoelectric Peltier device. In another embodiment, the thermoelectric Peltier device may be used in combination with an opthalmic ointment such as, for example, erythromycin, neosporin, amoxicillin or the like, or a combination thereof.

In one embodiment the skin care benefit is cosmetic. In another embodiment the skin care or other benefit is therapeutic and optionally cosmetic. The term “cosmetic” as used herein refers to serving an aesthetic purpose in treating or beautifying the skin and/or improving the appearance thereof, while the term “therapeutic” refers to the treatment of an ailment, to cure or restore to health, or to reduce or ameliorate one or more symptoms of an ailment. In this regard, in one embodiment, a successful treatment includes any degree of reduction or amelioration of the severity of an ailment or a single symptom thereof, from the point of view of the user, and need not effect a complete cure or elimination of all or even one symptom. Similarly, successfully achieving the aesthetic purpose in treating or beautifying the skin is, in one embodiment, according to the point of view of the user and, in another embodiment, according to the point of view of an objective observer.

In one embodiment, the skin is human skin. The skin may include one or more of the face, mouth, nose, eyes, lips, neck, hands, feet, elbows, knees, chin, throat, cheeks, chest, nipples, buttocks, genitals, or a combination thereof. In one embodiment, the skin care benefit includes any benefit to any layer of the skin including one or more of epithelium, stratum corneum, epidermis, dermis, lower dermis, or any combination thereof.

In another embodiment, the benefit includes any benefit to any part of the body, e.g., muscle, joint, extremity, head, torso, chest, back, arm, leg, hand, foot, neck, forehead, bone, ligament, tendon, genital, and the like, or any combination thereof. To treat or obtain the benefit in these parts of the body, the thermoelectric Peltier device may be suitably applied (or contacted) with an area near or in the immediate vicinity of that body part.

In one embodiment, the method further includes cleaning the skin, which can be carried out before, after or during the contacting and cooling steps, or a combination of these. After application, the composition may be optionally wiped or washed off or otherwise removed from the skin. Alternatively, the composition may be allowed to remain on the skin. In one embodiment, the composition is allowed to remain on the skin for a period of time after the cooling and contacting steps, for example, overnight, and then washed or wiped off or otherwise removed.

In another embodiment, the composition is left on the skin after the contacting and cooling steps for a time ranging from a few minutes to greater than one day. This range includes all values and subranges therebetween, including 1, 2, 3, 4, 5, 10, 15, 20, 25, 30, 35, 40, 45, 50, 55, 60, and 90 minutes, 2, 4, 6, 8, 10, 12, 14, 16, 18, 20, 22, and 24 hours, or longer.

In another embodiment, the composition is left on the skin after the contacting and cooling for about 1 to 12 hours.

In another embodiment, the contacting and contacting and cooling steps are repeated on a daily basis.

The method can be carried out periodically, for example, in one or more of the morning, afternoon, evening, or any combination thereof, for a period of time until the desired skin benefit or other benefit, as determined by the user, is achieved. The method may be carried out every day, every other day, or on a weekly or other basis. In one embodiment, the method is carried out every morning and afternoon. In another embodiment, the method is carried out every evening before bedtime. The period and frequency of application is easily determined by the user or the user's cosmetologist, beauty professional, aesthetician, or dermatologist as appropriate.

The amount and frequency of carrying out the method may vary widely depending upon factors such as the particular skin or other benefit desired, the severity of any skin or other ailment, the location and/or type of skin or body part involved, the user's skin or body part sensitivity, and the degree of treatment desired. Given the teachings herein combined with the knowledge of one of ordinary skill in the art, one can easily utilize the kit herein and carry out the method to its full scope.

In one embodiment, the thermoelectric Peltier device is applied to the skin or body part for a short period of time, then withdrawn therefrom for another short period of time, then reapplied for a short period of time, and so on. The respective periods of time may suitably and respectively range from about 1 second to about an hour. This range includes all values and subranges therebetween, including 1, 2, 3, 4, 5, 6, 7, 8, 9, 10, 15, 20, 25, 30, 35, 45, 50, 55, and 60 seconds, 1, 2, 3, 4, 5, 6, 7, 8, 9, 10, 15, 20, 25, 30, 35, 40, 45, 50, 55, and 60 minutes, and any combination thereof. In another embodiment, the application may carried out once a day or several times a day, and optionally be carried out over successive days or until the desired treating effect or benefit is obtained.

In one embodiment, the kit and method are utilized in a spa, salon, medical office or other facility specializing in skin or body care. In another embodiment, the kit and method are utilized at home.

The composition is not particularly limited, so long as it is suitable for application to the skin. In one embodiment, the composition is cosmetically and/or dermatologically acceptable. Here, it is intended that the terms cosmetically and dermatologically acceptable mean that the composition and/or any of its constituent components are suitable for application to skin without undue toxicity, incompatibility, instability, allergic response, irritation, discomfort, or the like. In one embodiment, the composition and/or any of its constituent components are of sufficiently high purity or quality that they do not cause undue toxicity, incompatibility, instability, allergic response, irritation, discomfort, or the like.

As used herein, the term “topical application” means to apply or spread the composition onto the surface of the skin. In one embodiment, the composition may be applied directly to the skin. In another embodiment, the composition may be applied first to the skin-contacting surface of the thermoelectric Peltier device, and then contacted with the skin.

The composition may be in any form suitable for skin application. For example, the composition may be in the form of a liquid, cream, serum, paste, emulsion, gel, balm, lotion, milk, foam, semi-solid, ointment, stick, O/W emulsion, W/O emulsion, and the like, or any combination thereof.

The composition may suitably include any ingredient, active or adjuvant as appropriate, in any amount, so long as it is suitable for application to the skin. Suitable ingredients, actives and adjuvants, which are not intended to be limiting, may be found in the CTFA International Cosmetic Ingredient Dictionary and Handbook, 11th Edition (2006) or “Remington: The Science and Practice of Pharmacy”, 21st Edition, the entireties of each of which are hereby incorporated by reference.

For example, the composition may optionally include one or more of skin protectant, solubilizing agent, antibiotic, emollient, moisturizer, thickener, emulsifier, neutralizer, gelling agent, analgesic, anaesthetic agent, anti-acne agent, anti-bacterial agent, anti-yeast agent, anti-fungal agent, anti-viral agent, anti-dandruff agent, anti-dermatitis agent, anti-pruritic agent, anti-metic agent, anti-inflammatory agent, anti-hyperkeratolytic agent, anti-perspirant, anti-psoriatic agent, anti-seborrheic agent, anti-aging agent, anti-wrinkle agent, antioxidant, de-pigmenting agent, skin sensation agent, anti-foaming agent, anti-microbial agent, humectant, powder, astringent, surfactant, pH regulating agent, UV absorbing agent, UV protecting agent, tanning agent, astringent, absorbent, binder, biological additive, denaturant, chelating agent, preservative, reducing agent, skin bleaching agent, solvent, retinyl acetate, retinyl palmitate, ascorbyl palmitate, magnesium ascorbyl palmitate, magnesium ascorbyl phosphate, tocopherol, vitamin C, vitamin E, vitamin A, vitamin A palmitate, vitamin E acetate, vitamin K, vitamin F, Retin A (Tretinoin), RESTYLANE™, hyaluronic acid, Juvederm™, Radiesse™, adapalene, retinol, echinacea, hormone, retinoid, sphingosine, sphingosine derivative, cyclopentasiloxane, dimethiconal, cyclomethicone, dimethicone, squalane, glycerin, steareth-20, palmitoyl tetrapeptide 3, saccharomyces copper ferment, saccharomyces manganese ferment, saccharomyces zinc ferment, safflower, carthamus tinctorium oil, mineral oil, caprylic triglyceride, capric triglyceride, propylene glycol, glyceryl stearate, PEG-100 stearate, saccharomyces zinc ferment, stearic acid, hydroxyethylcellulose, carbomer, triethanolamine, lavendula angustifolia, methylparaben, diazolidinyl urea, zinc, hydroxyacid, alpha-hydroxy acid, glycyrrhetinic acid, seaweed derivative, salt water derivative, phytoanthocyanin, epigallocatechin 3-gallate, collagen, phytonutrient, botanical product, herbaceous product, enzyme, mineral, insulin, trace element (including ionic calcium, magnesium, etc), Rogaine, a hair growth stimulating agent, a hair growth inhibiting agent, natural or synthetic melanin, metalloproteinase inhibitor, proline, hydroxyproline, chlorophyll, copper chlorophyllin, carotenoid, bacteriochlorophyll, phycobilin, carotene, xanthophyll, anthocyanin, fragrance, pigment, colorant, essential oil, clove oil, menthol, camphor, eucalyptus oil, eugenol, menthyl lactate, bisabolol, witch hazel distillate, green tea extract, water, and the like, or a combination thereof.

The amounts of the constituent components described herein are not particularly limited.

The composition may include an aqueous base, oily base, oil-in-water emulsion base, water-in-oil emulsion base. Non-limiting examples of these include vegetable oil, animal oil, synthetic oil, fatty acid, natural glyceride, synthetic glyceride, volatile silicone oil, nonvolatile silicone oil, water, aqueous alcohol, glycol, acetone, ethanol, aqueous alcohol, glycol, ethylene glycol, propylene glycol, butane-1,3 diol, isopropyl myristate, isopropyl palmitate, mineral oil, and the like, or a combination thereof.

The composition may include one or more emollients or moisturizers. Non-limiting examples of these include saturated or unsaturated fatty acids, saturated or unsaturated fatty alcohols, saturated or unsaturated fatty acid esters, fats, waxes, oils, synthetic oils, natural oils, mineral oils, vegetable oils, animal oils, alkylene glycol ethers, alkylene glycol esters, polyalkylene glycol ethers, polyalkylene glycol esters, isopropyl myristate, cetyl palmitate, octyldodecylmyristate, octyl palmitate, isostearyl neopentanoate, isocetyl stearyl stearate, isocetyl alcohol, beeswax, behenyl alcohol, cetyl alcohol, petrolatum, squalene, fatty sorbitan esters, lanolin, lanolin derivatives, lanolin alcohol, ethoxylated lanolin, hydroxylated lanolin, acetylated lanolins, cholesterol, cholesterol derivative, glycerin, maleated vegetable oil, animal triglycerides, vegetable triglycerides, almond oil, peanut oil, wheat germ oil, linseed oil, jojoba oil, oil of apricot pits, walnut oil, palm nut oil, pistachio nut oil, sesame seed oil, rapeseed oil, cade oil, corn oil, peach pit oil, poppyseed oil, pine oil, castor oil, soybean oil, avocado oil, safflower oil, coconut oil, hazelnut oil, olive oil, grapeseed oil, sunflower seed oil, diisostearylmalate, diisostearyldimerate, triisostearyltrimerate, guanidine, aloe vera, polyhydroxy alcohols, sorbitol, glycerol, hexanetriol, propylene glycol, hexylene glycol, polyethylene glycol, sugars, starches, hyaluronic acid, urea, and the like, or a combination thereof.

The composition may include one or more surfactants or emulsifying agents. Some examples of surfactants and emulsifying agents include ceteareths, ceteths, cetyl alcohol, deceths, dodoxynols, glyceryl palmitate, glyceryl stearate, laneths, myreths, nonoxynols, octoxynols, oleths, laureth 23, PEG 20 stearate, PEG-castor oil, poloxamers, poloxamines, polysorbates, ammonium laureth sulfate and sodium laureth sulfate, mink-amidopropyl dimethyl 2-hydroxyethylammonium chloride, and the like, or a combination thereof.

In one embodiment, the composition may include one or more gelling agents. Non-limiting examples of gelling agents include water soluble or colloidally water soluble polymers and include cellulose ethers, such as hydroxyethyl cellulose, methyl cellulose, hydroxypropylmethyl cellulose, polyvinylalcohol, polyquaternium-10, guar gum, hydroxypropyl guar gum, xanthan gum, acrylic acid/ethyl acrylate copolymers, carboxyvinyl polymers, maleic anhydride-alkyl methylvinylethers and copolymers, natural gums, polymethacrylate copolymer, oleogels, trihydroxystearin, aluminum magnesium hydroxy stearate, and the like, or a combination thereof.

In one embodiment, the composition optionally includes two or more ingredients selected from the group consisting of cyclopentasiloxane, dimethiconal, cyclomethicone, dimethicone, squalane, glycerin, steareth-20, palmitoyl tetrapeptide 3, saccharomyces copper ferment, saccharomyces manganese ferment, saccharomyces zinc ferment, ascorbyl palmitate, safflower, carthamus tinctorium oil, mineral oil, caprylic triglyceride, capric triglyceride, propylene glycol, glyceryl stearate, PEG-100 stearate, saccharomyces zinc ferment, stearic acid, hydroxyethylcellulose, carbomer, triethanolamine, lavendula angustifolia, methylparaben, diazolidinyl urea, vitamin E, zinc, hydroxyacid, alpha-hydroxy acid, glycyrrhetinic acid, and water.

Some examples of particularly suitable compositions include those available from Farr Laboratories such as PEAU DE SOIE™ Essence Complex, PEAU DE SOIE™ Face and Neck Crème, PEAU DE SOIE™ Eye Serum, FRĒO™ Bio-Activating Eye Serum, FRĒO™ Therapeutic Essence Complex, and FRĒO™ Intensive Anti-Aging Eye Crème.

In one embodiment, the composition includes cyclopentasiloxane, dimethiconal, cyclomethicone, dimethicone, squalane, glycerin, steareth-20, palmitoyl tetrapeptide 3, saccharomyces copper ferment, saccharomyces manganese ferment, saccharomyces zinc ferment, and ascorbyl palmitate. Such a composition would be suitable, for example, as an essence complex.

In one embodiment, the composition includes cyclopentasiloxane, dimethiconol, cyclomethicone, dimethicone, squalane, glycerin, steareth-20, palmitoyl tetrapeptide 3, saccharomyces copper ferment, saccharomyces manganese ferment, saccharomyces zinc ferment, and ascorbyl palmitate. Such a composition would be suitable, for example, as an eye serum.

In one embodiment, the composition includes water, safflower (carthamus tinctorium) oil, mineral oil, caprylic/capric triglyceride, propylene glycol, glyceryl stearate, PEG-100 stearate, dimethicone, saccharomyces copper ferment, saccharomyces manganese ferment, saccharomyces zinc ferment, glyccrin, steareth-20, palmitoyl tetrapeptide 3, stearic acid, hydroxyethylcellulose, carbomer, triethanolamine, lavendula angustifolia, methylparaben, and diazolidinyl urea. Such a composition would be suitable, for example, as a face and neck cream.

In one embodiment, the composition includes cyclopentasiloxane, dimethiconal, cyclomethicone, palmitoyl oligopeptide, palmitoyl tetrapeptide-7, chrysin, water, glycerin, steareth-20 hydroxysuccinimide, allantoin, calendula officinalis flower extract, chamomilla recutita (matricaria) flower extract, ascorbyl palmitate, dimethicone, and squalane. Such a composition would be suitable, for example as a bio-activating eye serum.

In one embodiment, the composition includes cyclopentasiloxane, dimethiconal, cyclomethicone, palmitoyl oligopeptide, palmitoyl tetrapeptide-7, glycerin, water, butylene glycol, carbomer, polysorbate 20, allantoin, calendula officinalis flower extract, chamomilla recutita (matricaria) flower extract, ascorbyl palmitate, dipalmitoyl hydroxyproline, and dimethicone. Such a composition would be suitable, for example as a therapeutic essence complex.

In one embodiment, the composition includes water, cyclopentasiloxane, dimethicone copolymer, glycerin, carthamus tinctorius (safflower) seed oil, butylene glycol, carbomer, polysorbate 20, palmitoyl oligopeptide, palmitoyl tetrapeptide-7, cetyl alcohol, steareth-20, hydroxysuccinimide, chrysin, stearic acid, polyacrylate-13, polyisobutene, phenoxyethanol, methylparaben, ethylparaben, butylparaben, propylparaben, mica, silica, PEG-8 beeswax, allantoin, calendula officinalis flower extract, chamomilla recutita (matricaria) flower extract, gynostemma pentaphyllum leaf extract, tetrasodium EDTA, and triethanolamine. Such a composition would be suitable, for example, as an intensive anti-aging eye crème.

The composition may contain Matrixyl 3000™, Haloxyl™, or both, which are available from Sederma.

The composition may include one or more UV agents, for example, benzophenone-3, benzophenone-4, oxytyl dimethyl PABA, octyl methoxy cinnamate, octyl salicylate, octocrylene, p-methylbenzylidene camphor, butyl methoxy dibenzoyl methane, titanium dioxide, zinc oxide, and the like, or a combination thereof.

In one embodiment, the composition may include one or more preservatives. Non-limiting examples of preservatives include sodium benzoate, imidazolidinyl urea, diazolidinyl urea, phenoxyethanol, methylparaben, ethylparaben, propylparaben, and the like, or a combination thereof.

The composition may include one or more skin bleaching or exfoliating agents. Non-limiting examples of these include hydroquinone, kojic acid, sodium metabisulfite, hydroxy carboxylic acids, keto acids, hydroxybenzoic acids, alpha-hydroxy acids, salicylic acid, beta hydroxy acids, sulfonated phenol, glycolic acid, lactic acid, malic acid, tartaric acid, citric acid, ascorbic acid, mandelic acid, azelaic acid, glyceric acid, tartronic acid, gluconic acid, benzylic acid, pyruvic acid, ethyl pyruvate, 2-hydroxybutyric acid, salicylic acid, mineral acids, non-hydroxy acids, trichloracetic acid, and the like, or a combination thereof.

The composition may include one or more anti-acne agents. Non-limiting examples of these include sulfur, resorcinol, pyruvic acid, retinoids, retinoic acid, retinoic acid derivatives, antimicrobials, benzoyl peroxide, erythromycin, tetracycline, triclosan, azelaic acid, clindamycin, chlorhexidine, tetracycline, neomycin, miconazole, clotrimazole, and the like, or a combination thereof.

The composition may include one or more chelating agents, for example, disodium EDTA, tetrasodium EDTA, sodium metasilicate, and the like, or a combination thereof.

The composition may optionally include one or more external analgesics, for example, aspirin, nonsteroidal antiinflammatories, and the like, or a combination thereof.

The composition may include one or more steroidal antiinflammatory drugs, for example, hydrocortisone and the like.

The composition may include one or more antifungals, for example, mycostatin, clotrimazole, myconazole, chlorophyll, carotenoids and the like, or a combination thereof.

The composition may be suitably prepared according to conventional methods such as those described in, for example, “Bennett's Cosmetic Formulary”, 2nd Edition (1992) or “Remington: The Science and Practice of Pharmacy”, 21st Edition, the entire contents of each of which are hereby incorporated by reference.

In one embodiment, the composition is used in combination with a thermoelectric Peltier device, which is configured to contact the composition and to cool the skin.

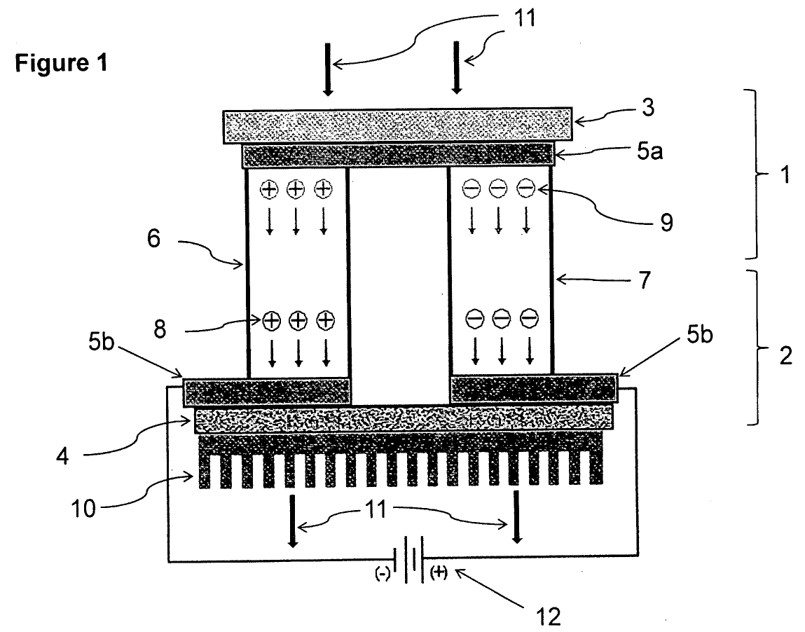

The Peltier effect is well-known. The thermoelectric Peltier device contains one or more thermoelectric modules. Thermoelectric modules are solid-state heat pumps that operate on the Peltier effect. Thermoelectric (sometimes referred to herein as “TE”) modules are known and are commercially available. As such, they are not particularly limited. One embodiment of a thermoelectric module is shown in FIG. 1.

In one embodiment of the thermoelectric Peltier device, shown in FIG. 1, the module shown has a cool side 1 and a hot side 2. The cool side includes a “cool” surface or thermally conductive substrate 3; the hot side includes a “hot” surface or thermally conductive substrate 4. These thermally conductive substrates 3 and 4 are not particularly limited, and may be the same or different material so long as they are thermally conductive. For example, the substrates 3 and 4 may be ceramic, metal or polymer, or any other suitable thermally conductive material, or a combination thereof.

In the embodiment shown in FIG. 1, one or more cool side electrical conductors 5a and one or more hot side electrical conductors 5b are present. The thermally conductive substrates 3 and 4 are in thermal contact with, but electrically insulated from, the respective electrical conductors 5. The electrical conductors 5 are not particularly limited and may be any suitable design, material, and configuration known in the art.

In the embodiment shown in FIG. 1, an array of p- and n-type semiconductor elements 6 and 7 are present and in electrical contact with electrical conductors 5. As shown in FIG. 1, the p- and n-type semiconductor elements 6 and 7 are electrically connected in series and thermally connected in parallel.

When the thermoelectric module is powered up, electron holes 8 and electrons 9 move from the cool side 1 to the hot side 2 in the respective p-type and n-type semiconductors 6 and 7. Heat is transferred from the cool side 1 to the hot side 2 in the process, described in more detail, which is not by way of limitation, below.

When electrons 9 move from the hot side 2 of the n-type semiconductor 7 into the hot side conductor 5b, they drop down into a lower energy level. Heat is released in this process. The electrons 9 move freely in the hot side conductor 5b. When the electrons 9 leave the hot side conductor 5b and enter the hot side 2 of the p-type semiconductor 6, they fill electron holes 8 in order to move through the p-type semiconductor 6. When the electrons 9 fill electron holes 8, the electrons 9 drop down into a lower energy level, and heat is released in the process. As the electrons 9 move from the hot side 2 to the cool side 1 of the p-type semiconductor 6, the electron holes 8 in the p-type semiconductor 6 move from the cool side 1 to the hot side 2. For the electrons 9 to move from the p-type semiconductor 6 into the cool side conductor 5a, the electrons 9 must bump up into a higher energy level, and heat is absorbed in the process. The electrons 9 move freely through the cool side conductor 5a until they reach the cool side 1 of the n-type semiconductor 7. For the electrons 9 to move into the n-type semiconductor 7, they must bump up into a higher energy level; heat is absorbed when this occurs; and the cycle begins anew.

In the above-described cycle, the electrical charge carriers (holes 8 in the p-type semiconductor 6; electrons 9 in the n-type semiconductor 7) travel from the cold side 1 to the hot side 2, heat is absorbed at the cool side 1 and released at the hot side 2 of the thermoelectric module. By this cycle, which is driven by the power supply 12, heat is transferred from the cool side 1 to the hot side 2. The direction of heat flow is shown by arrows 11. A heat sink 12 is present to conduct heat away from the thermoelectric module.

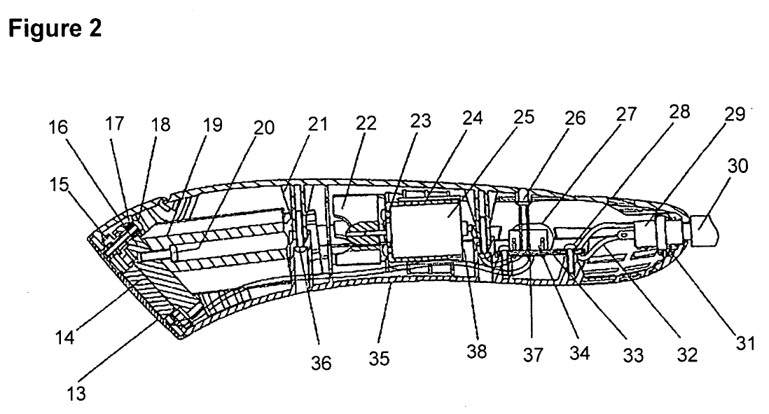

The thermoelectric Peltier device, which contains one or more thermoelectric modules, may be configured to comfortably fit in the hand of the user, for use with a stand, or for use as a tabletop device. In one embodiment, the device is configured for handheld use such as shown in FIG. 2. In one embodiment, the device has one or more cool plates 13. The cool plate 13 may be in thermal contact with the cool side 1 or the cool surface 3 of the thermoelectric module, or the cool plate 13 may itself form the cool surface or substrate 3 of the thermoelectric module. In one embodiment, the thermoelectric Peltier device is configured for contacting the composition and for cooling the skin. In this embodiment, the outer surface of cool plate 13 presents a skin-contacting surface for contacting the user's skin.

One embodiment of a thermoelectric Peltier device configured for handheld use is shown in FIG. 2. There, a cool plate 13 is configured to contact the skin of the user. The cool plate 13 is in thermal contact with a cool surface or substrate 3 (not shown) of a thermoelectric (TE) module 14. The thermoelectric (TE) module 14 is held by a TE module holder 15. The hot surface or substrate 4 (not shown) is in thermal contact with heat sink plate A 16. A washer 17 and screw 18 connect the cool plate 13, TE module holder 15 and heat sink plate A 16. Heat sink plate A 16 is in thermal contact with heat sink plate B 19, being connected thereto by a screw 20. Upper cabinet 21 is configured to hold a fan 22 connected to a motor 25. A screw 23 holds the motor mount 24 and motor 25. An LED 26 shows when the device is powered and/or ready for use. A switch knob 27 connected to switch 34 is present for powering up the device and/or adjusting the temperature of cool plate 13. A screw 28 holds the electronic controller 33, printed circuit board, or the like to the lower cabinet 35. A DC jack 29 is held by DC jack holder 31 and is connected to adaptor 30 for supplying power via internal wire 32 to the device. Resistor 37 and capacitor 38 are present for electrical operation. A screw 36 connects lower cabinet 35 to upper cabinet 21. The upper and lower cabinets 35 and 21 may be conveniently made of any material, such as ABS or other plastic. The cool plate 13 and heat sink plates A B 16 and 19 may be suitably be made of any thermally conductive material, such as aluminum, ceramic, stainless steel, and the like, or any combination thereof.

In one embodiment, the thermoelectric Peltier device comprises a skin-contacting surface having a temperature of about 20° F. to about body temperature. This range includes all values and subranges therebetween, including about 20, 22, 24, 26, 28, 30, 32, 34, 36, 38, 40, 42, 44, 45, 46, 48, 50, 55, 60, 65, 70, 75, 80, 85, 90, 91, 92, 93, 94, 95, 96, 97, 98, less than body temperature, body temperature, and 99° F. For purposes herein, body temperature is the temperature of a healthy human, or about 98.6° F.

In one embodiment, the thermoelectric Peltier device comprises a skin-contacting surface having a temperature of about 30° F. to about body temperature. In another embodiment, the thermoelectric Peltier device comprises a skin-contacting surface having a temperature of about 30° F. to less than body temperature.

In one embodiment, the thermoelectric Peltier device comprises a skin-contacting surface having a temperature of about 40° F. to about body temperature. In another embodiment, the thermoelectric Peltier device comprises a skin-contacting surface having a temperature of about 40° F. to less than body temperature.

The thermoelectric Peltier device may suitably operate at a power range of about 2 to about 10 W. This range includes all values and subranges therebetween, including 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5 and about 10 W.

Suitable thermoelectric Peltier devices may be obtained from, for example, Watronix, Inc.

In one embodiment, the temperature of the skin-contacting surface is pre-set, i.e., it is not variable. In an alternate embodiment, the temperature of the skin-contacting surface is variable, i.e., it may be adjusted by the user. In one embodiment, the temperature of the skin-contacting surface can reach 45° F. in two minutes or less.

In one embodiment, the thermoelectric Peltier device comprises a skin-contacting surface that is smooth, textured, abrasive, or a combination thereof. For example, the skin-contacting surface may be the outer surface of cool plate 13, such as shown in FIG. 2. In another embodiment, a removable pad, cover, sock, or sleeve may be utilized over the outer surface of cool plate 13, so long as the pad or cover is thermally conductive. The pad, cover, sock, or sleeve (not shown) may be made of any soft or textured or abrasive material such as a cloth, plastic, or metal, or a combination thereof. It may be disposable or reusable, for example, by washing. The pad, cover, sock, or sleeve may be configured for wet or dry use. For example, the pad, cover, sock, or sleeve may be wet with liquid, gel, cream, paste, or the like, and contacted with the skin or body part to heat or cool the skin or body part as appropriate.

In one embodiment, the thermoelectric Peltier device is configured for contacting the composition and for cooling the user's skin. In another embodiment, the thermoelectric Peltier device is configured for contacting the composition and for heating the user's skin. In another embodiment, the thermoelectric Peltier device may be used to heat and/or cool the skin or body part without the use of a composition.

The thermoelectric Peltier device may be battery powered or plug into an electrical outlet. If plugged into an outlet, the device may be configured to plug directly into an outlet, or may be configured for use with an external AC adaptor. If battery powered, the battery may be rechargeable, if desired.

In one embodiment, the thermoelectric Peltier device is configured to heat the skin. In this embodiment, the thermoelectric Peltier device may additionally contain a heating device (not shown), e.g., a resistive heater in thermal contact with the skin-contacting surface of the thermoelectric Peltier device. Alternatively, or additionally, the heating device may be one or more thermoelectric modules similar to those shown in FIG. 1, but which are reversed, i.e., wherein the module is configured such that the hot side 2 and/or hot surface or substrate 4 presents a skin-contacting surface or is configured to be in thermal contact with the skin-contacting surface. Alternatively, or additionally, the heating device may be achieved by reversing the current flow from that shown in FIG. 1. In such an embodiment, the respective flows of electron holes 8, electrons 9, and heat flow 11 are reversed, the cool side 1 and cool surface or substrate 3 becomes hot, and the hot side 2 and the hot surface or substrate 4 becomes cool. With the heating device, in one embodiment, the composition is used in combination with the thermoelectric Peltier device, which is configured to contact the composition and to heat the skin. With the heating device, in another embodiment, the composition is used in combination with a thermoelectric Peltier device, which is configured to contact the composition and to alternately heat and cool the skin.

In one embodiment, with the heating device, the thermoelectric Peltier device comprises a skin-contacting surface having a temperature of about 40° F. to about 110° F. This range includes all values and subranges therebetween, including about 40, 42, 44, 45, 46, 48, 50, 55, 60, 65, 70, 75, 80, 85, 90, 91, 92, 93, 94, 95, 96, 97, 98, less than body temperature, body temperature, 99, 100, 105, and about 110° F. For purposes herein, body temperature is the temperature of a healthy human, or about 98.6° F.

In one embodiment, the thermoelectric Peltier device has a skin-contacting surface having a temperature range of about 40° F. to about body temperature.

In another embodiment, the thermoelectric Peltier device has a skin-contacting surface having a temperature range of about body temperature to about 110° F.

In one embodiment, the thermoelectric Peltier device or skin-contacting surface thereof is configured to vibrate to provide the user with a massage at the affected skin area or body part or to enhance the beneficial effect of the composition. A vibration module (not shown) might include a reciprocating bar driven by a magnet, or an eccentrically weighted rotor.

In one embodiment, the handheld device is configured such that it can be easily converted between handheld use and use with a stand or other holder.

In another embodiment, the thermoelectric Peltier device is in the form of a form-fitting mask, for example, configured to fit or generally fit the user's face or other body part of interest. In such an embodiment, the thermoelectric Peltier device may contain a plurality of thermoelectric modules disposed about various points in the mask. The skin- and composition-contacting surface of the mask, not shown, is cooled by the plurality of thermoelectric modules.

### EXAMPLES

The following example is provided for purposes of illustration only, and is not intended to be limiting unless otherwise specified.

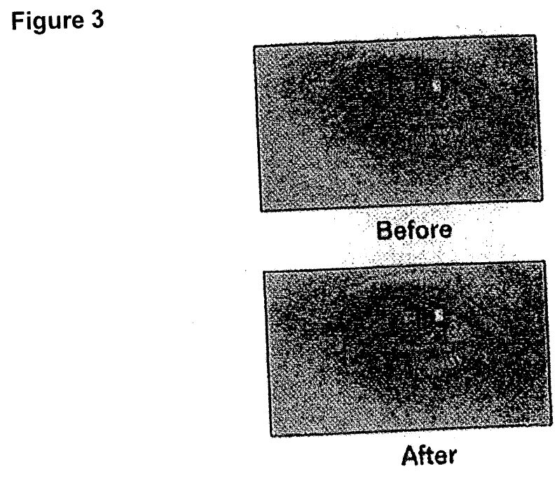

A female subject was observed to have dark circles, puffiness, and fine lines in the skin area around her eyes. This is shown in the “before” photograph in FIG. 3. An eye cream composition was applied to the affected area. The composition and affected area were contacted and cooled with the thermoelectric Peltier device for about 45 seconds. After the treatment, the appearance of dark circles, puffiness, and fine lines was observed to be reduced. This is shown in the “after” photograph in FIG. 3.

### KEY TO FIGURES

1 cool side
2 hot side
3 cool surface or substrate
4 hot surface or substrate
5 electrical conductors (5a cool side conductor; 5b hot side conductor)
6 p-type semiconductor
7 n-type semiconductor
8 electron holes
9 electrons
10 heat sink
11 heat flow direction
12 power supply and circuit
13 cool plate
14 thermoelectric (TE) module
15 TE module holder
16 heat sink plate A
17 washer
18 screw
19 heat sink plate B
20 screw
21 upper cabinet
22 fan
23 screw
24 motor mount
25 motor
26 LED
27 switch knob
28 screw
29 DC jack
30 adaptor
31 DC jack holder
32 internal wire
33 electronic controller
34 switch
35 lower cabinet
36 screw
37 resistor
38 capacitor

What is claimed is:

1. A method of treating the skin or body part, comprising:
contacting the skin or body part with a composition; and
contacting the composition and heating and/or cooling the skin or body part with a thermoelectric Peltier device.

2. The method of claim 1, wherein the skin is human skin selected from the group consisting of face, mouth, nose, eyes, lips, neck, hands, feet, elbows, knees, chin, throat, cheeks, chest, nipples, buttocks, genitals or a combination thereof.

3. The method of claim 1, wherein the treating provides a skin care benefit to one or more of epithelium, stratum corneum, epidermis, dermis, lower dermis, or any combination thereof.

4. The method of claim 1, wherein the body part is selected from the group consisting of muscle, joint, extremity, head, torso, chest, back, arm, leg, hand, foot, neck, forehead, bone, ligament, tendon, genital, and the like, or any combination thereof.

5. The method of claim 1, wherein the skin or body part is in need of the treating.

6. The method of claim 1, wherein the treating provides a skin care or other benefit selected from the group consisting of reducing or eliminating the appearance of fine lines and wrinkles, retarding or reducing the noticeable effects of skin aging, retarding or reducing the appearance of dark circles around the eyes, retarding or reducing the appearance of puffiness around the eyes, treating or reducing the effects of at least one of the following: skin wrinkling, sagging, dryness, irritation, inflammation, swelling, aging, photodamage, bruising, dandruff, acne, keratosis, eczema, flakiness, age spots, hyper-pigmented skin, inflammatory dermatosis, psoriasis, calluses, sunburn, candida, thrush, burn injury, atopic dermatitis, rosacea, fibroblast collapse, low collagen production, skin blemishes, corns, boils, bursas, styes, or a combination thereof.

7. The method of claim 1, wherein the treating provides a skin care or other benefit selected from the group consisting of aiding in improving skin texture, improving skin smoothness, improving skin firmness, improving skin tone, promotion of smooth and supple skin, improving skin elasticity, moisturizing skin, improving the radiance, clarity and general youthful appearance of skin, improving skin color, improving skin hydration, increasing flexibility, firmness, smoothness, suppleness and elasticity of the skin, improving a skin connective tissue, re-stretching collapsed fibroblasts, interfering with collagen breakdown, enhancing collagen production, enhancing elastin production, cooling the skin, cooling elastin and/or collagen, reduce swelling of bursas, exfoliating, detaching and shedding of superficial epithelial cells, improving the thickness of skin, enhancing skin repair and healing, or a combination thereof.

8. The method of claim 1, wherein the treating provides a benefit selected from the group consisting of treating or reducing pain, discomfort, or irritation in one or more parts of the body, or reducing swelling in one or more parts of the body, reducing pain, discomfort, or irritation in one or more parts of the body caused by, or reducing swelling in one or more parts of the body caused by any one or more of surgical procedure, bodily injury, bruising, hair waxing or removal, menopause, hot flashes, headache, nausea, laser treatment, microdermabrasion, exfoliation or other skin treatment, carpel tunnel syndrome, temporomandibular joint (TMJ) disorder, sports injury, overuse injury, muscle soreness, or a combination thereof.

9. The method of claim 1, wherein the thermoelectric Peltier device comprises a skin-contacting surface having a surface temperature of 40° F. to about 110° F.

10. The method of claim 1, wherein the composition comprises two or more ingredients selected from the group consisting of cyclopentasiloxane, dimethiconal, cyclomethicone, dimethicone, squalane, glycerin, steareth-20, palmitoyl tetrapeptide 3, saccharomyces copper ferment, saccharomyces manganese ferment, saccharomyces zinc ferment, ascorbyl palmitate, safflower, carthamus tinctorium oil, mineral oil, caprylic triglyceride, capric triglyceride, propylene glycol, glyceryl stearate, PEG-100 stearate, saccharomyces zinc ferment, stearic acid, hydroxyethylcellulose, carbomer, triethanolamine, lavendula angustifolia, methylparaben, diazolidinyl urea, vitamin E, zinc, hydroxyacid, alpha-hydroxy acid, glycyrrhetinic acid, and water.

11. The method of claim 1, wherein the composition is one or more of the following (a), (b), or (c):
(a) an essence complex comprising cyclopentasiloxane, dimethiconal, cyclomethicone, dimethicone, squalane, glycerin, steareth-20, palmitoyl tetrapeptide 3, saccharomyces copper ferment, saccharomyces manganese ferment, saccharomyces zinc ferment, and ascorbyl palmitate;
(b) an eye serum comprising cyclopentasiloxane, dimethiconol, cyclomethicone, dimethicone, squalane, glycerin, steareth-20, palmitoyl tetrapeptide 3, saccharomyces copper ferment, saccharomyces manganese ferment, saccharomyces zinc ferment, and ascorbyl palmitate; or
(c) a face and neck cream comprising water, safflower or carthamus tinctorium oil, mineral oil, caprylic/capric triglyceride, propylene glycol, glyceryl stearate, PEG-100 stearate, dimethicone, saccharomyces copper ferment, saccharomyces manganese ferment, saccharomyces zinc ferment, glyccrin, steareth-20, palmitoyl tetrapeptide 3, stearic acid, hydroxyethylcellulose, carbomer, triethanolamine, lavendula angustifolia, methylparaben, and diazolidinyl urea.

12. The method of claim 1, wherein the composition and skin or body part is heated with the thermoelectric Peltier device.

13. The method of claim 1, wherein the composition and skin or body part is cooled with the thermoelectric Peltier device.

14. A method of treating the skin or body part, comprising:
contacting the skin or body part and heating and/or cooling the skin or body part with a thermoelectric Peltier device.

15. The method of claim 15, wherein the skin or body part is heated with the thermoelectric Peltier device.

16. The method of claim 15, wherein the skin or body part is cooled with the thermoelectric Peltier device.

17. A skin or body care kit, comprising:
a composition suitable for application to the skin or body part; and
a thermoelectric Peltier device configured to contact the composition and to heat and/or cool the skin or body part.

18. The kit of claim 17, wherein the composition is one or more of the following (a), (b), or (c):
(a) an essence complex comprising cyclopentasiloxane, dimethiconal, cyclomethicone, dimethicone, squalane, glycerin, steareth-20, palmitoyl tetrapeptide 3, saccharomyces copper ferment, saccharomyces manganese ferment, saccharomyces zinc ferment, and ascorbyl palmitate;
(b) an eye serum comprising cyclopentasiloxane, dimethiconol, cyclomethicone, dimethicone, squalane, glycerin, steareth-20, palmitoyl tetrapeptide 3, saccharomyces copper ferment, saccharomyces manganese ferment, saccharomyces zinc ferment, and ascorbyl palmitate; or
(c) a face and neck cream comprising water, safflower or carthamus tinctorium oil, mineral oil, caprylic/capric triglyceride, propylene glycol, glyceryl stearate, PEG-100 stearate, dimethicone, saccharomyces copper ferment, saccharomyces manganese ferment, saccharomyces zinc ferment, glyccrin, steareth-20, palmitoyl tetrapeptide 3, stearic acid, hydroxyethylcellulose, carbomer, triethanolamine, lavendula angustifolia, methylparaben, and diazolidinyl urea.

19. The kit of claim 17, wherein the composition comprises two or more ingredients selected from the group consisting of cyclopentasiloxane, dimethiconal, cyclomethicone, dimethicone, squalane, glycerin, steareth-20, palmitoyl tetrapeptide 3, saccharomyces copper ferment, saccharomyces manganese ferment, saccharomyces zinc ferment, ascorbyl palmitate, safflower, carthamus tinctorium oil, mineral oil, caprylic triglyceride, capric triglyceride, propylene glycol, glyceryl stearate, PEG-100 stearate, saccharomyces zinc ferment, stearic acid, hydroxyethylcellulose, carbomer, triethanolamine, lavendula angustifolia, methylparaben, diazolidinyl urea, vitamin E, zinc, hydroxyacid, alpha-hydroxy acid, glycyrrhetinic acid, and water.

20. The kit of claim 17, wherein the thermoelectric Peltier device comprises a skin-contacting surface and is configured such that the skin-contacting surface has a surface temperature of about 40° F. to about 110° F.

21. The kit of claim 17, wherein the thermoelectric Peltier device is configured to contact the composition and to cool the skin or body part.

22. The kit of claim 17, wherein the thermoelectric Peltier device is configured to contact the composition and to heat the skin or body part.

23. A thermoelectric Peltier device, comprising:
at least one surface configured to contact the skin or body part and heat and/or cool the skin or body part.
